(19) United States
(12) Patent Application Publication
(10) Pub. No.: US 2016/0215236 A1
(43) Pub. Date: Jul. 28, 2016

(54) COMPOSITION COMPRISING CELLS AND A POLYUNSATURATED FATTY ACID HAVING AT LEAST 20 CARBON ATOMS (LC-PUFA)

(71) Applicant: DSM IP ASSETS B.V., Heerlen (NL)

(72) Inventors: Daniel VERKOEIJEN, Stewartsville, NJ (US); Hendrik Louis BIJL, Vlaardingen (NL); Kristian ZUUR, HK Delfgauw (NL)

(21) Appl. No.: 14/472,312

(22) Filed: Aug. 28, 2014

Related U.S. Application Data
(63) Continuation of application No. 13/504,155, filed on Nov. 19, 2012, now abandoned.
(60) Provisional application No. 61/257,772, filed on Nov. 3, 2009.

(30) Foreign Application Priority Data
Nov. 22, 2009 (EP) PCT/EP2009/065592
Nov. 22, 2009 (EP) PCT/EP2009/065593

(51) Int. Cl. C11B 5/00; C12N 15/82; A23D 9/007; C11C 1/08

(52) U.S. Cl. CPC C11B 5/0085; C11C 1/08; C12N 15/8247; A23L 1/296; A23L 1/3006; A23D 9/007; A23V 2002/00

(57) ABSTRACT

The present invention relates to a composition comprising a polyunsaturated fatty acid having at least 20 carbon atoms (LC-PUFA) and cells, which composition has a thermal induction time (T.I.T.) of >24 hours at 40° C. The invention also relates to a process for drying a composition comprising cells and a LC-PUFA, the process comprising drying the composition at a temperature of below 40° C.

The present invention relates to a composition comprising cells and a polyunsaturated fatty acid having at least 20 carbon atoms (LC-PUFA), to a process for drying a composition comprising cells and a LC-PUFA, and to a process for obtaining an LC-PUFA or an oil containing an LC-PUFA from a composition comprising calls and an LC-PUFA.

LC-PUFAs can be produced by micro-organisms in a fermentation process. LC-PUFAs can also be produced in plants. The microorganisms or plant parts containing the LC-PUFA can then be pre-treated after which LC-FUFA or oil containing the LC-PUFA can be isolated.

For instance, WO 2006/085672, describes a process wherein an LC-PUFA is isolated from a microbial biomass. Wet cells are dried in a two-stage drying process. Drying temperatures of 120° C. and higher are used, and dried cells having a moisture content of 1-2 wt. % are obtained.

It is possible to isolate the LC-PUFA and/or oil containing the LC-PUFA from the LC-PUFA-containing composition immediately after its production. However, in practice the LC-PUFA-containing composition is often stored and/or transported before further use such as isolation of the LC-PUFA and/or oil containing the LC-PUFA.

It is now found that LC-PUFA-containing compositions are susceptible to self-heating. Viz. during storage, the temperature can increase spontaneously, ultimately resulting in unexpected explosions and fires. It is further found that this susceptibility increases with increasing LC-PUFA content and with increasing number of double bonds of the LC-PUFAs.

It is an object of the invention to provide a composition comprising (i) LC-PUFA and (ii) cells, which composition is safer.

The invention now provides a composition comprising (i) a polyunsaturated fatty acid having at least 20 carbon atoms (LC-PUFA) and (ii) cells, which composition has a thermal induction time (T.I.T.) of >24 hours at 40° C.

The composition according to the invention has the advantage that its safety is improved, and that the risk of spontaneous temperature increase, unexpected explosions and fires is decreased. A further advantage of the composition of the invention is that storage of the composition does not negatively affect the quality of the LC-PUFA or oil containing the LC-PUFA, or at least imparts the quality to a lesser extent.

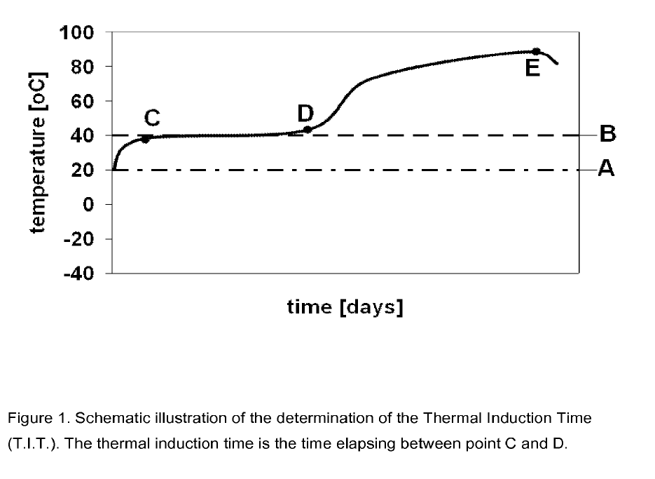

The composition according to the invention has a thermal induction time (T.I.T.) of >24 hours at 40° C. The T.I.T. of the composition is determined using a heat accumulation storage test as described in Recommendations on the Transport of Dangerous Goods, Manual of Tests and Criteria, Section 28.4.4, Test H.4, United Nations, New York, 1999, with the following adaptations and specifications: A glass 0.5 l Dewar vessel with an internal diameter of 57 mm and a height of 210 mm is used. The heal loss of the Dewar vessel is 16 mW/K. The sample size is 75% of the volume of the Dewar vessel. The Dewar vessel is dosed with a rubber stopper with a height of approximately 50 mm, loosely tightened to allow for respiration. A thermocouple is inserted in the Dewar vessel through a hole in the centre of the stopper. The Dewar vessel containing the sample, which have an initial temperature of 20° C., are placed in a chamber that is controlled at 40° C., and the temperature of the sample is monitored using the thermocouple. The thermal induction time is defined as time elapsing between moment at which the temperature of the sample reaches the temperature of 2° C. below the chamber temperature (hence 38° C.), and the moment at which the temperature of the sample reaches the temperature of 2° C. above the chamber temperature (hence 42° C.). The determination of the T.I.T. is illustrated in FIG. 1.

Preferably the com position according to the invention has a T.I.T. of at least 2 days (48 hours), preferably at least 3 days (72 hours), preferably at least 4 days (96 hours), preferably at least 5 days, measured at 40° C. The T.I.T. may he at least 8 days, for instance at least 10 days, measured at 40° C. There is no specific upper limit for the T.I.T. The T.I.T. may be less than 25 days, for instance less than 20 days, measured at 40° C.

The composition having the increased T.I.T. according to the invention can be obtained based on the teaching provided by the invention.

The composition may be a dried composition. It is found that the T.I.T. increases if the drying temperature is decreased. It is further found that it is advantageous to decrease the drying temperature if the content of LC-PUFAs in the composition or the number of double bonds of the LC-PUFAs is higher.

For instance, the drying temperature may be below 40° C., preferably below 35° C., more preferably below 33°, more preferably below 30° C., more preferably below 25° C. As used herein, the drying temperatures refer to the temperature of the product in the dryer. For instance, if the drier is a fluid bed dryer, the drying temperature refers to the temperature of the bed.

Accordingly, the invention also provides a process for drying a composition comprising cells and LC-PUFA, the process comprising drying the composition at a temper slurs of below 40° C., preferably below 35° C., preferably below 33° C., preferably below 30° C., preferably below 25° C.

The drying may be by any suitable method. Drying may be performed in any suitable dryer. Preferably a dryer is used which prevents or minimizes the formation of hot spots. In a preferred embodiment drying is effected using a fluid bed dryer.

It is found that the T.I.T. increases with decreasing drying time.

In a preferred embodiment, drying is effected using conditioned air. Preferably air is used having a dew point of <15° C., preferably <10° C., preferably <5° C., Decreasing the dew point has the advantage that preferred moisture contents can efficiently be achieved at preferred (low) drying temperatures.

Accordingly, the invention also provides a process for drying a composition comprising cells and a LC-PUFA, the process comprising contacting the composition with conditioned air, which preferably has a dew point of <15° C., preferably <10° C., preferably <5° C. Preferably, drying is effected at the preferred drying temperatures mentioned above.

It is further found that the T.I.T. increases with increases if the moisture content of the composition increases. Preferably the (e.g. dried) composition has a moisture content of at least 1 wt. %, preferably at least 2 wt. %, preferably at least 3 wt. %, preferably at least 4 wt. %. There is no specific upper limit for the moisture content. The composition may have a moisture content of below 20 wt. %, for instance below 15 wt. %, for instance below 12 wt. %, for instance below 10 wt. %, for instance below 9 wt %. Decreasing the moisture content below the preferred values is found to increase the microbial stability of the composition.

As used herein, the moisture content is calculated on a wet weight basis, i.e. on the basis of the total weight of the composition (including dry matter, oil, and moisture). It and can be determined by the skilled person, for instance by evaporating the water at a temperature of 105° C., and determining the weight of the evaporated moisture.

In a preferred embodiment of the invention, drying of the composition as disclosed herein, results in a preferred moisture content as disclosed herein.

It is further found that avoiding the formation of free radicals during processing of the composition can result increased values for the T.I.T. Based on this insight, the skilled person can avoid steps resulting in the formation of free radicals. Accordingly, it is generally preferable to minimize exposure of the cells to circumstances that can promote the formation of free radicals, e.g. exposure to high temperatures end/or to oxygen.

If the cells are microbial cells, advantageously a fermentation broth containing the cells is heated such as to sufficiently kill off the enzymes that may be present in the fermentation broth. Preferred heating protocols are described in WO 97/037032 and WO 2004/001021 which are hereby incorporated by reference. Preferably a fermentation broth having a low dissolved oxygen content, for instance <10 ppm, for instance <5 ppm, for instance <2 ppm, for instance <1 ppm, is heated. Killing off the enzymes. In particular using the protocols as referred to hereinabove, may result in increased values of the T.I.T.

In a preferred embodiment, the composition according to the invention has art oil content of at least 10 wt. %, for instance at least 20 wt. %, for instance at least 30 wt. %, for instance at least 40 wt. %. The oil content may be below 70 wt. %, for instance below 80 wt. %. The oil content may foe determined by methods known to the skilled person. A suitable method for determining the oil content of the composition as used herein is by using a Soxhlet extraction using n-hexane as the solvent, wherein the composition subjected to the extraction has a moisture content <15 wt. % and wherein the composition and cells are comminuted (to ensure that ail oil is released from the cells and can dissolve into the solvent). As used herein, the oil content is calculated on a dry basis, i.e. on the basis of the total dry weight of the composition (including dry matter and oil, but excluding moisture).

In a preferred embodiment, the composition according to the invention has an oil content as defined above, wherein the composition of the oil is as in the preferred embodiments described below.

In a preferred embodiment, the composition comprises an oil which comprises at least 10 wt. %, for instance at least 20 wt. %, for instance at least 30 wt. %, for instance at least 40 wt. % of PUFAs with at least 3 double bonds with respect to the total fatty acids in the oil, for instance below 80 wt. %, for instance below 70 wt. %, for instance below 80 wt. % of PUFAs with at least 3 double bends with respect to the total fatty acids in the oil. As used herein, the wt. % of PUFAs with at least 3 double bonds refers to the sum of all PUFAs with at least 3 double bonds.

In a preferred embodiment, the composition comprises an oil which comprises at least 10 wt. %, for instance at least 20 wt. %, for instance at least 30 wt. %, for instance at least 40 wt. % of arachidonic acid (ARA) with respect to the total fatty acids in the oil, for instance below 80 wt. %, for instance below 70 wt. %, for instance below 60 wt. % ARA with respect to the total fatty acids in the oil.

In a preferred embodiment, the composition comprises an oil which comprises at least 10 wt. %, for instance at least 20 wt. %, for instance at least 30 wt. %, for instance at least 40 wt. % of docosahexaenoic acid (DHA) with respect to the total fatty acids in the oil, for instance below 80 wt. %, for instance below 70 wt. %, for instance below 80 wt. % DHA with respect to the total fatty acids in the oil.

A suitable method for determining the composition of the oil as used herein is to extract the oil from the composition using the Soxhlet extraction using n-hexane as described hereinabove, and to determine the fatty acid composition of the extracted oil.

It is preferred to select lower drying temperatures and shorter residence times in the dryer if the oil content and/or number of double bonds is relatively high.

Based on the teaching herein it is possible to high values for the T.I.T. even for compositions with a high oil content and/or for compositions containing an oil with a high concentration of PUFAs with at least 3 double bonds.

As used herein, the following abbreviations are used throughout the entire application:

PUFA refers to a polyunsaturated fatty acid

LC-PUFA (long chain polyunsaturated fatty acid) refers to a PUFA having at least 20 carbon atoms

HUFA (highly unsaturated fatty acid) refers to a PUFA having at least three double bonds

LC-PUFA (long chain highly unsaturated fatty acid) refers to a polyunsaturated fatty acid having at least 20 carbon atoms and at least three double bonds.

The invention is not limited to a specific LC-PUFA. In an embodiment of the invention, the LC-PUFA has at least three double bond. In a further embodiment of the invention, the LC-PUFA has at least four double bonds. The benefits of the invention are even more pronounced for LC-PUFAs having an increasing number of double bonds, as the susceptibility to self beating increases with increasing number of double bonds.

The LC-PUFA may be an ω-3 LC-PUFA or an ω-6 LC-PUFA

LC-PUFAs include for instance:

dihomo-γ-linolenic acid (DGLA, 20:3 ω-6)

arachidonic acid (ARA, 20:4 ω-6)

eicosapentaenoic acid (EPA, 20:5 ω-3)

docosapentaenoic acid (DPA, 22:5 ω-3, or DPA 22:5, ω-6),

docosahexaenoic acid (DHA: 22:6 ω-3);

Preferred LC-PUFAs include arachidonic add (ARA) and docosahexaenoic acid (DHA). In particular ARA is preferred.

The composition according to the invention comprises cells. The cells may be any cells containing and/or having produced the LC-PUFA.

In an embodiment of the invention, the cells are microbial cells (microorganisms).

Examples of microbial cells are yeast cells, bacterial cells, fungal cells, and algal cells. Fungi are preferred, preferably of the order Mucorales. Example are Mortierella, Phycomyces, Blakeslea, Aspergillus, Thraustochytrium, Pythium or Entomophthora. The preferred source of arachidonic acid (ARA) is from Mortierella alpina. Algae can be dinoflagellate and/or include Porphyridium, Nitszchia, or Crypthecodinium (e.g. Crypthecodium cohnii). Yeasts include those of the genus Pichia or Saccharomyces, such as Pichia ciferii. Bacteria can be of the genus Propionibacterium.

In an embodiment of the invention, the composition comprises a fungus of the genus Mortierella, preferably of the species Mortierella alpina, wherein preferably the LC-PUFA is ARA or DGLA.

In an embodiment of the invention the composition comprises a fungus of the order Thraustochytriales, for instance from the genus Thrauslochytrium or Schizochytrium, and wherein preferably the LC-PUFA is DHA and/or EPA.

In an embodiment of the invention, the composition comprises an algae of the genus Crypthecodinium, preferably of the species Crypthecodinium cohnii, wherein preferably the LC-PUFA is DHA.

In another embodiment of the invention, the cells are plant cells. The cells may be plant cells of a transgenic plant.

Suitable plants and seeds are for instance described in WO 2005/083093, WO 2008/009600, and WO 2009/130291, the contents of which are hereby incorporated by reference. Other plants and seeds that can fee used in the invention are for instance disclosed in WO 2008/100545, WO 2008/124806, WO 2008/124048, WO 2008/128240, WO 2004/071467, WO 2005/059130, the contents of which are hereby incorporated by reference. The seeds may be (transgene) soybeans or (transgene) canola seeds. The plant may be a (transgene) soybean plant or a (transgene) canola plant.

In a preferred embodiment, the plant is a (transgenic) plant of the family Brassicaceae, for instance the genera Brassica, Camelina, Melanosinapis, Sinapis, Arabidopsis, for example the genera and species Brassica alba, Brassica carinata, Brassica hirta, Brassica napus, Brassica rapa ssp., Sinapis arvensis, Brassica juncea, Brassica juncea var. juncea, Brassica juncea var. crispifolia, Brassica juncea var. follosa, Brassica nigra, Brassica sinapioides, Camelina sativa, Melanosinapis communis, Brassica oleracea or Arabidopsis thaliana.

The composition may be any biomass comprising a LC-PUFA. Preferably, the composition is a (dried) composition obtained or obtainable by a drying process disclosed herein.

The composition may be a microbial biomass comprising a microorganism and a LC-PUFA. Preferred microorganisms and LC-PUFAs are mentioned hereinabove.

In a possible embodiment of the invention a composition comprising microorganisms (microbial cells) according to the invention is obtained in a process comprising heating (also referred to as pasteurization or sterilization) a fermentation broth comprising the microbial cells, dewatering the microbial cells, e.g. by filtration, and drying the microbial cells in a process described hereinabove. In a preferred embodiment, the dewatered microbial cells are granulated prior to drying, preferably by extrusion. Preferably granulation, e.g. extrustion is performed at a temperature below 25° C. A preferred process is described in WO 97/037032 which is herewith incorporated by reference.

In an embodiment of the invention, the composition comprises seeds comprising an LC-PUFA and/or the composition may be in the form of seeds, said seeds having a thermal induction time (T.I.T.) of >24 hours at 40° C. Preferably, the seeds are seeds of plants mentioned hereinabove.

It is found that a keeping the keeping the percentage of damaged seeds low, results in an increased T.I.T.

Preferably, less than than 12% of the seeds are total damaged seeds, preferably less than 8%, preferably less than 5%, preferably less than 3% of the seeds are total damaged seeds.

Preferably, less than 8% of the seeds are distinctly green seeds, preferably less than 4%, preferably less than 2%. preferably less than 1% of the seeds are distinctly green seeds.

Preferably, less than 0.5% of the seeds are heated seeds, preferably less than 0.3%, preferably less than 0.1%, preferably less than 0.05% of the seeds are heated seeds.

In a preferred embodiment, less than 8% of the seeds are total damaged seeds, less than 4% of the seeds are distinctly green seeds, and less than 0.3% of the seeds are heated seeds, in another preferred embodiment, less than 5% of the seeds are total damaged seeds, less than 2% of the seeds are distinctly green seeds, and less than 0.1% of the seeds are heated seeds. In another preferred embodiment, less than 3% of the seeds are total damaged seeds, less than 1% of the seeds are distinctly green seeds, and less than 0.05% the seeds are heated seeds.

As used herein the percentages of total damaged seeds, distinctly green seeds and heated seeds are determined in accordance with the Official Grain Grading Guide. 2001 of the Canadian Grain Commission (for Canola and Rapeseed)

Seeds having the preferred percentages of total damaged seeds, distinctly green seeds and/or heated seeds can foe obtained by appropriate selection of the seeds after harvest.

In a further aspect of the invention there, the invention provides seeds comprising an LC-PUFA, and which have percentages of total damaged seeds, distinctly green seeds and/or heated seeds as disclosed hereinabove.

Preferably, the seeds comprise at least 5 wt. %, preferably at least 10 wt. %, preferably at least 15 wt. %, preferably at least 20 wt. % of an LC-PUFA (for instance an LC-PUFA as described herein, with respect to the total fatty acids in the seeds.

Preferably, the seeds comprise at least 5 wt. %, preferably at least 10 wt. %, preferably at least 15 wt. %, preferably at least 20 wt. % of an ω-6 LC-PUFA, with respect to the total fatty acids in the seeds.

Preferably, the seeds comprise at least 5 wt. %, preferably at least 10 wt. %, preferably at least 15 wt. %, preferably at least 20 wt. % of ARA, with respect to the total fatty acids in the seeds.

Preferably, the seeds comprise at least 5 wt. %, preferably at least 10 wt. %, preferably at least 15 wt. %, preferably at least 2.0 wt. % of an ω-3 LC-PUFA, with respect to the total fatty acids in the seeds.

Preferably, the seeds comprise at least 5 wt. %, preferably at least 10 wt. %, preferably at least 15 wt. %, preferably at least 20 wt. % of DHA, with respect to the total fatty acids in the seeds.

Preferably, the seeds comprise less than 2 wt. % of erucic acid, preferably less than 1 wt. %, preferably less than 0.5 wt. % based on the total fatty acids in the seeds.

The composition according to the invention may suitably be stored prior to further use and/or processing.

Advantageously, the composition is stored at a temperature of below 10° C., preferably below 5° C., preferably below 0° C., preferably below minus 5° C., preferably below minus 10° C. There is no specific lower limit for the storage temperature. Generally, the composition is stored at a temperature of above minus 30° C.

If the composition comprises seeds or is in the form of seeds, preferably the seeds have a moisture content of less than 15 wt. %, for instance less than 12 wt. %, for instance less than 10 wt. %, for instance less 9,5 wt. %, for instance above 6 wt. %, for instance above 7 wt. %, for instance above 8 wt. %. The moisture content may for instance be between 6 and 15 wt. %, for instance between 7 and 12 wt. %. for instance between 8 and 10 wt. %. The preferred moisture contents can be obtained by drying the seeds as described hereinabove.

The composition may be stored for any suitable period. The composition may for instance be stored for at least 1 day, for instance at least 1 week, for instance at least 2weeks, for instance at least 1 months, for instance at least 3 months. There is no specific upper limit for the storage period. The composition, may for instance be stored for less than 12 months, for instance less than 6 months.

The invention further comprises a process for obtaining a LC-PUFA or an oil comprising a LC-PUFA, comprising isolating said LC-PUFA or oil comprising a LC-PUFA from a composition according the invention or from a composition obtained or obtainable by the process according to the invention.

The lipid LC-PUFA or oil comprising the LC-PUFA may be obtained by extraction the LC-PUFA or oil comprising the LC-PUFA from the composition, preferably by solvent extraction. Any suitable solvent may be used, for instance a C₁₋₁₀ alkyl ester (e.g. ethyl or butyl acetate), toluene, a C₁₋₃ alcohol (e.g. methanol, propanol), a alkanes (e.g. hexane) or a supercritical-fluid (e.g. liquid CO₂ or supercritical propane). Preferably, the solvent is a non-polar solvent, for instance a C₃-C₆ alkane (preferably hexane) or a supercritical fluid (preferably supercritical CO₂ or supercritical propane). Preferred extraction procedures are described in WO 97/037032.

If the composition comprises seeds or is in the form of seeds, the LC-PUFA and/or oil comprising the LC-PUFA may be isolated as follows.

The seeds and/or the composition comprising the seeds may be crushed and/or flaked. This may facilitate recovery of the LC-PUFA or oil containing the LC-PUFA. The crushed and/or flaked seeds and/or composition comprising the seeds may then be heated, for instance at a temperature above 80 Heating may he at a relatively low temperature. The seeds and/or composition comprising the seeds may for instance be heated at a temperature between 50 and 90° C., for instance between 60 and 80° C., preferably for a period of between 2 to 60 minutes, preferably between 5 to 30 minutes, if an increased temperature is selected, the duration of the heating is preferably decreased.

The seeds and/or composition comprising the seeds may be heated at a high rate. The process may for instance comprise heating the seeds or composition comprising the seeds, whereby the temperature passes from 40 to 70° C. in less than 1 minute, preferably less than 30 seconds, preferably less than 20 seconds. The process may for instance comprise heating the seeds and/or composition comprising the seeds, whereby the temperature passes from 40 to 100° C. in less than 1 minute, preferably less than 30 seconds, preferably less than 20 seconds.

The process according to the invention may comprises heating the seeds and/or composition comprising the seeds using superheated steam. The process according to the invention may for instance comprise contacting the seeds and/or composition comprising the seeds with superheated steam.

Preferably, the process according to the invention comprises heating the seeds and/or composition comprising the seeds at a relatively high temperature, e.g. between 120 and 160° C. for a relatively short period. The process may for instance comprise maintaining the seeds and/or composition comprising the seeds at a temperature above 120° C., for instance below 160° C., for a period of less than 8 minutes, for instance less than 5 minutes, for instance less than 3 minutes, for instance less than 2 minutes. Maintaining the seeds and/or composition comprising the seeds at a temperature between 120, for instance below and 160° C. may be for a period of at least 5 seconds, preferably at least 10 seconds.

Preferably, the seeds and/or composition comprising the seeds is cooled at a relatively high rate. Preferably, the temperature of the seeds and/or composition comprising the seeds is decreased from the maximum temperature to a temperature of 40° C. in less than 80 minutes, preferably less than 30 minutes, preferably less than 15 minutes.

The protocols may be used separately or in combination. For instance, the heating at a high rate may be combined with maintaining the seeds or composition comprising the seeds at a preferred temperature for a relatively short period and/or with a rapid cooling rate.

The heating is not limited to a specific stage of the process. The beating may be effected prior to or after any comminuting of (e.g. crushing or flaking) the seeds. In a further aspect, the invention provides a process for heating seeds comprising a LC-PUFA, whereby the seeds are heated as disclosed hereinabove.

A fraction of oil may be obtained by pressing the seeds or composition comprising the seeds. Pressing the seeds such as to expel a fraction of oil may be performed using methods known in the art. A screw press may be used. In a preferred embodiment, the invention comprises pressing the seeds or composition comprising the seeds to expel oil using a press, for instance a screw press, which is cooled,

A further fraction of oil may be obtained by solvent extraction from press cake obtained after pressing described hereinabove.

Purifying of the oil may comprise degumming, refining, bleaching and/or deodorizing. These are known steps, and can be carried out by the skilled person. In a preferred embodiment, deodorizing is effected at a temperature below 200° C., preferably below 190° C., preferably below 185° C. Decreasing the deodorization temperature to below the preferred values improves the quality of the oil.

The invention further provides a process for obtaining a food product, in particular an infant formula, comprising obtaining a LC-PUFA or an oil comprising a LC-PUFA from the composition according to the invention, and incorporating said LC-PUFA or oil comprising said LC-PUFA in said food product.

Further preferred aspects, embodiments and features are disclosed in the claims

Preferred features and characteristics of one embodiment and/or aspect of the invention are applicable to another embodiment mutatis mutandis. As used herein, the preferred features and characteristics of the LC-PUFA apply to the LC-PUFAs in all aspects and embodiments of the invention.

The invention is further disclosed with reference to the following examples without being limited thereto.

### EXAMPLES

### Example 1

Fermentation broth of Mortierella alpina, obtained after 8 days of fermentation was pasteurized at 70° C. for 1 hour. The pasteurized broth was filtered, resulting in a filter cake have a moisture content of 50 wt. %. The filter cake was crumbled and extruded at a temperature below 15° C. The extrodate (diameter 3 mm) was dried in a continuous fluid bed drier with three zones to a moisture content of 7%. In the first zone the bed temperature was 32° C. and the air temperature 50° C. (T_{dew point}=15° C.).

1^{st} zone: bed temperature 32° C., the air temperature 50° C. (T_{dew point}=150° C.): 45 minutes

2^{nd} zone: bed temperature 32° C., air temperature 35° C. (T_{dew point}=1° C.): 45 minutes

3^{rd} zone: bed temperature 15° C., air temperature 15° C. (T_{dew point}1° C.): 30 minutes

The oil content of the dried biomass was 39%. The ARA content was 46% with respect to the total fatty acids in the oil.

Thermal induction time (T.I.T.) measured at 40° C.: 9 days.

### Comparative Experiment A

The fermentation and pasteurization is repeated. The wet cells are recovered using a continuous dehydrator and disrupted, and then drying is carried out by hot air drying (hot air temperature 120° C.) with a vibrating fluidized bed dried to a moisture content of 1 wt. %. The dried cells are cooled by supplying room temperature air in the fluidized bed. ARA and oil content are as in example 1.

Thermal induction time (T.I.T.) measured at 40° C. ; <12 hours

### Example 2

Seeds containing 19% Aracbidonio acid (with respect to total fatty acids) are obtained from transgenic Brassica plants that are transformed using the procedures described in WO2008009600.

The seeds have the following specifications (determined in accordance with the Official Grain Grading Guide, 2001 of the Canadian Grain Commission): distinctly green <2%, total damaged <5%.

The seeds, having a moisture content of 17 wt. %, are dried using a fluid bed drier. The bed temperature is 28° C. Conditioned air is used having a dew point of 10° C. The dried seeds have a moisture content of 8.5 wt. %. The oil content is 35 wt. %.

Thermal induction time (T.I.T.) measured at 40° C.: 14 days.

### DESCRIPTION OF THE FIGURE

FIG. 1 shows a Schematic illustration of the determination of the Thermal Induction Time (T.I.T.).
A. Initial temperature of the sample
B. Chamber temperature
C. Sample temperature 2° C. below the chamber temperature
D. Sample temperature 2° C. above the chamber temperature

1. Composition comprising (i) a polyunsaturated fatty acid having at least 20 carbon atoms (LC-PUFA) and (ii) cells, which composition has a thermal induction time (T.I.T.) of >24 hours at 40° C.

2. Composition according to claim lf which has a moisture content of between 1 and 20 wt. %, preferably between 2 and 15 wt. %, preferably between 3 and 12 wt. %, preferably between 3.5 and 10 wt. %, preferably between 4 and 9 wt. %.

3. Composition according to claim 1, which has an oil content of at least 10 wt. %, for instance at least 20 wt. %, for instance at least 30 wt. %, for instance at least 40 wt. %, for instance below 70 wt. %, for instance below 60 wt. %.

4. Composition according to claim 1, wherein the composition comprises an oil which comprises at least 10 wt. %, for instance at least 20 wt. %, for instance at least 30 wt. %, for instance at least 40 wt. % of PUFAs with at least 3 double bonds with respect to the total fatty acids in the oil, for instance below 80 wt. %, for instance below 70 wt. %, for instance below 60 wt. % of PUFAs with at least 3 double bonds with respect to the total fatty acids in the oil.

5. Composition according to claim 1, wherein the LC-PUFA has at least 3 double bonds.

6. Composition according to claim 1, wherein the LC-PUFA is an ω-3 or an ω-6 PUFA.

7. Composition according to claim 1, wherein the LC-PUFA is selected from dihomo-γ-linolenic acid (DGLA, 20:3 ω-6), arachidonic acid (ARA, 20:4 ω-6), eicosapentaenoic add (EPA, 20:5 ω-3), docosahexaenoic acid (DHA: 22:6 ω-3), docosapentaenoic acid (DPA 22:5 ω-3, or DPA 22:5, ω-6).

8. Composition according to claim 1, wherein the LC-PUFA is ARA or DHA.

9. Composition according to claim 1, wherein the cells are microbial cells.

10. Composition according to claim 9, wherein the microbial cells are yeast cells, bacterial cells, fungal cells, or algal cells.

11. Composition according to claim 1, wherein the composition comprises a microorganism of the genus Mortierella, preferably of the species Mortierella alpina, and wherein preferably the LC-PUFA is ARA.

12. Composition according to claim 1, wherein the composition comprises a microorganism of the order Thraustochytriales, for instance of the genus Thraustochytrium or Schizochytrium, and wherein preferably the LC-PUFA is DHA or EPA.

13. Composition according to claim 1, wherein the composition comprises a microorganism of the genus Crypthecodinium, preferably of the species Crypthecodinium cohnii, and wherein preferably the LC-PUFA is DHA.

14. Composition according to claim 1, wherein the cells are plant cells.

15. Composition according to claim 14, wherein the cells are plant cells of a transgenic plant.

18. Composition according to claim 14, wherein the cells are plant cells of a plant of the family Brassicaceae, preferably of the genus Brassica.

17. Process for drying a composition comprising cells and a LC-PUFA, the process comprising drying the composition at a temperature of below 40° C., preferably below 35° C., preferably below 33 preferably below 30° C., preferably below 25° C.

18. Process, for instance according to claim 17, for drying a composition comprising cells and a LC-PUFA, the process comprising contacting the composition with conditioned air which preferably has a dew point of ≦15° C., preferably <10° C. preferably <5° C.

19. Process according to claim 17, comprising drying the composition using a fluid bed dryer.

20. Composition obtainable by the process according to any claim 17.

21. Composition according to claim 20, which has a moisture content.

22. Process for obtaining a LC-PUFA or an oil comprising a LC-PUFA, the process comprising isolating said LC-PUFA or oil comprising a LC-PUFA from a composition or from a dried composition obtained or obtainable by the process according to claim 17.

23. Process for obtaining a food product, in particular an infant formula, said process comprising obtaining a LC-PUFA or an oil comprising a LC-PUFA according to the process of claim 22, and incorporating said LC-PUFA or oil comprising said LC-PUFA in said food product.
